(19) United States
(12) Patent Application Publication
(10) Pub. No.: US 2023/0363623 A1
(43) Pub. Date: Nov. 16, 2023

(54) ENDOSCOPE

(71) Applicant: Japan Lifeline Co., Ltd., Tokyo (JP)

(72) Inventor: Yuki KODAMA, Tokyo (JP)

(21) Appl. No.: 18/356,794

(22) Filed: Jul. 21, 2023

Related U.S. Application Data
(63) Continuation of application No. PCT/JP2021/010708, filed on Mar. 16, 2021.

(51) Int. Cl. A61B 1/00; A61B 1/018; A61B 1/015

(52) U.S. Cl. CPC A61B 1/00068; A61B 1/018; A61B 1/015

(57) ABSTRACT

An object is to provide an endoscope in which opening and closing operations of a flow path of a suction tube can be easily performed while a handle is being held and operated. An endoscope of the disclosure includes a shaft including a forceps channel and water channels, a handle including a forceps port, a water port, and a suction port, a suction tube being inserted inside the handle from the suction port, extending inside the handle, and having a distal end portion joining a communication path between the forceps channel and the forceps port, and a water tube being inserted inside the handle from the water port, extending inside the handle, and having a lumen communicating with the water channels. The handle includes a valve mechanism that opens and closes a suction flow path of the suction tube.

### CROSS REFERENCE TO RELATED APPLICATIONS

This application is a continuation application of International Application No. PCT/JP2021/010708, filed on Mar. 16, 2021, the entire contents of which are hereby incorporated by reference.

### TECHNICAL FIELD

The disclosure relates to an endoscope, and more particularly to an endoscope including a shaft to be inserted into a body, an operation handle, and a camera.

### BACKGROUND

Conventionally, an endoscope including a shaft to be inserted into a body, an operation handle, and a camera has been known as a small endoscope used for diagnostic treatment of a biliary duct, a pancreatic duct, or the like via a duodenoscope (see JP 4764417).

Typically, the shaft constituting the endoscope is formed with a forceps channel also serving as a suction channel and a water channel.

The handle constituting the endoscope is provided with a forceps port, a water port, and a suction port.

The forceps port of the handle communicates with the forceps channel of the shaft.

A biopsy forceps inserted from the forceps port is inserted into the forceps channel of the shaft through the inside of the handle (a communication path between the forceps port and the forceps channel), and a distal end portion of a live body forceps extends out into a body from a forceps opening which is an opening of the forceps channel at a distal end of the shaft.

Usually, the forceps port of the handle also serves as a drug solution injection port.

By injecting a drug solution from the forceps port, the drug solution flows through the inside of the handle (the communication path) and the forceps channel of the shaft, flows out from the forceps opening, and is administered into the body.

The water port of the handle communicates with the water channel of the shaft.

Specifically, a water tube having a proximal end portion connected to a water bottle is inserted into the handle from the water port and extends inside the handle, and a distal end portion of the water tube is connected to the water channel of the shaft.

Water in the water bottle flows through the water tube and the water channel and flows out into the body from a water outlet which is an opening of the water channel at the distal end of the shaft. Accordingly, the distal end portion of the shaft and the surroundings thereof are cleaned, and fogging or the like of a camera lens is eliminated.

The suction port of the handle communicates with the forceps channel of the shaft and the forceps port (drug solution injection port).

Specifically, a suction tube having a proximal end portion connected to a suction pump is inserted into the handle from the suction port and extends inside the handle, and a distal end portion of the suction tube is connected to the communication path between the forceps channel and the forceps port.

By closing the forceps port and operating the suction pump, a body fluid or the like of a patient around the distal end portion of the shaft can flow into the forceps channel from the forceps opening, flow through the forceps channel and the suction tube, and flow out from the suction port.

### SUMMARY

When the drug solution is injected from the forceps port of the handle while the suction pump is operating, the injected drug solution is sucked by the suction tube connected to the communication path and cannot flow through the forceps channel of the shaft and flow out from the forceps opening.

In such a case, it is conceivable to stop the suction pump when injecting the drug solution from the forceps port, or to close a flow path of the suction tube extending out from the suction port to the outside of the handle and leading to the suction pump by using a two-way stopcock, a three-way stopcock, or the like.

However, it is not practical to stop the suction pump in injecting the drug solution.

In addition, it is troublesome for an operator who holds and operates the handle to close the flow path of the suction tube extending out from the handle.

The disclosure has been made in view of the above-described circumstances.

An object of the disclosure is to provide an endoscope in which opening and closing operations of a suction flow path of a suction tube can be easily performed while a handle is held and operated and a drug solution injected from a forceps port of the handle can be reliably flowed out from a forceps opening at a distal end of a shaft even while a suction pump is operating.
(1) An endoscope of the disclosure includes a shaft including a forceps channel and a water channel, the forceps channel also serving as a suction channel;
a handle disposed on a proximal end side of the shaft, the handle including a forceps port communicating with the forceps channel, a water port communicating with the water channel, and a suction port;
a camera disposed inside the shaft and the handle;
a suction tube having a proximal end portion connected to a suction unit, the suction tube being inserted inside the handle from the suction port and extending inside the handle, the suction tube having a distal end portion joining a communication path between the forceps channel and the forceps port; and
a water tube having a proximal end portion connected to a water supply unit, a tube being inserted inside the handle from the water port and extending inside the handle, the water tube having a lumen communicating with the water channel.

The handle includes a valve mechanism that opens and closes a suction flow path of the suction tube.

In the endoscope having such a configuration, because the handle includes the valve mechanism that performs opening and closing operations for the suction flow path of the suction tube, the opening and closing operations can be easily performed in a state where the handle is gripped.
(2) In the endoscope of the disclosure, the valve mechanism is preferably configured to be put into a “closed” state by closing the suction flow path by pressing an outer circumferential surface of the suction tube.
(3) In the endoscope according to (2) described above, preferably, the valve mechanism includes a tube retaining portion that is disposed inside the handle and retains the suction tube (a portion whose outer circumferential surface is to be pressed) extending inside the handle at a predetermined position, a cylinder portion that is attached to or formed at the handle and communicates between an inside and an outside of the handle, and a pressing member that moves along a direction of an axis of the cylinder portion while rotating around the axis, and
when the valve mechanism is in an “open” state, a distal end portion of the pressing member is located inside the cylinder portion, and when the valve mechanism is in the “closed” state, the distal end portion of the pressing member extends out from a distal end opening of the cylinder portion to an inside of the handle, and a distal end surface of the pressing member presses an outer circumferential surface of the suction tube retained by the tube retaining portion.

In the endoscope having such a configuration, by moving the pressing member constituting the valve mechanism in the “open” state along the direction of the axis of the cylinder portion while rotating the pressing member around the axis of the cylinder portion, the distal end portion of the pressing member located (waiting) inside the cylinder portion extends out from the distal end opening (an opening on an inner side of the handle) of the cylinder portion, and the distal end surface of the pressing member presses the outer circumferential surface of the suction tube retained by the tube retaining portion, whereby the suction flow path of the suction tube can be closed.
(4) In the endoscope according to (3) described above, preferably, the pressing member is formed by integrally molding a shaft portion including a proximal end portion extending out from a proximal end opening (an opening on an outer side of the handle) of the cylinder portion to an outside of the handle,
an operation lever that is attached to the proximal end portion of the shaft portion and causes a rotating operation of the pressing member,
a partial outer circumferential portion being formed to cover a part of an outer circumferential surface of the shaft portion and including an outer circumferential surface slidable with respect to an inner circumferential surface of the cylinder portion and a proximal end surface having a helical shape and being slidable with respect to an inner end surface (an inner end surface on a proximal end side) of the cylinder portion, and
the distal end portion, and
when the rotating operation of the shaft portion is caused by the operation lever, the pressing member moves along the direction of the axis of the cylinder portion while rotating around the axis by the proximal end surface of the partial outer circumferential portion sliding with respect to the inner end surface of the cylinder portion.
(5) In the endoscope according to (4) described above, preferably, the pressing member is configured to rotate substantially 90° around the axis of the cylinder portion while the valve mechanism is switched from the “open” state to the “closed” state.

In the endoscope having such a configuration, the valve mechanism can be switched from the “open” or “closed” state to the “closed” or “open” state by rotating the pressing member around the axis of the cylinder portion by 90° by using the operation lever.
(6) In the endoscopes according to (3) to (5) described above, preferably, the distal end portion of the pressing member has an annular shape.

In the endoscope having such a configuration, because a pressing force of the distal end surface of the pressing member onto the outer circumferential surface of the suction tube can be concentrated, the suction flow path of the suction tube can be more reliably closed.
(7) In the endoscopes according to (3) to (6) described above, preferably, the tube retaining portion includes a flat plate portion having a flat surface facing the distal end surface of the pressing member across the suction tube, and tube gripping portions formed on both sides of the flat plate portion.

In the endoscope having such a configuration, the suction tube (a portion whose outer circumferential surface is to be pressed) is gripped by the tube gripping portions formed on both sides of the flat plate portion, and the suction tube is sandwiched between the distal end surface of the pressing member and the flat surface of the flat plate portion, whereby the suction flow path of the suction tube can be more reliably closed.
(8) In the endoscope according to (7) described above, preferably, at least a portion of the suction tube retained by the tube retaining portion extends inside the handle in parallel with the water tube,
the tube retaining portion includes a partition wall portion including a protruding plate extending in a direction perpendicular to the flat plate portion, and
the suction tube and the water tube are spaced apart from each other by the partition wall portion.

In the endoscope having such a configuration, an outer circumferential surface of the water tube can be suppressed from being pressed by the distal end surface of the pressing member.

In the endoscope of the disclosure, the opening and closing operations of the suction flow path of the suction tube can be easily performed in a state where the handle is gripped.

Accordingly, when the drug solution is injected from the forceps port of the handle while the suction pump is operating, the valve mechanism is put into the “closed” state, whereby the injected drug solution can reliably flow out from the forceps opening at the distal end of the shaft.

### BRIEF DESCRIPTION OF DRAWINGS

FIG. 1 is an explanatory diagram illustrating the outer appearance of an endoscope of the disclosure.

FIG. 2 is an explanatory diagram illustrating the interior of a handle of the endoscope illustrated in FIG. 1.

FIG. 3 is a partially enlarged view (detailed view of portion III) illustrating a distal end portion of the endoscope illustrated in FIG. 1.

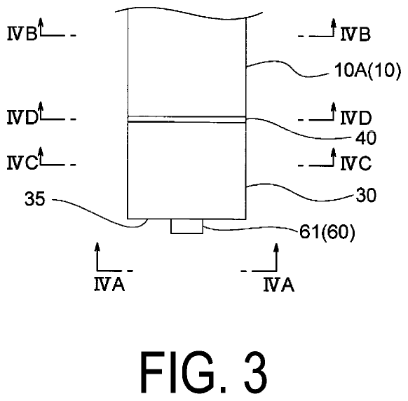

FIG. 4A is a view taken along the line IVA-IVA in FIG. 3.

FIG. 4B is a cross-sectional view taken along the line IVB-IVB in FIG. 3 (a cross-sectional view of a shaft).

FIG. 4C is a cross-sectional view taken along the line IVC-IVC in FIG. 3 (a cross-sectional view of a distal end tip).

FIG. 4D is a cross-sectional view taken along the line IVD-IVD in FIG. 3 (a cross-sectional view of an intermediate member).

FIG. 5 is a cross-sectional view schematically illustrating the interior of a camera head.

FIG. 6A is a perspective view illustrating the distal end portion of the endoscope illustrated in FIG. 1 (a camera is not illustrated).

FIG. 6B is a perspective view illustrating the distal end portion of the endoscope illustrated in FIG. 1 (the camera is not illustrated).

FIG. 7A is a cross-sectional view illustrating a state where a slide member of a camera connector constituting the endoscope illustrated in FIG. 1 is at a proximal end position.

FIG. 7B is a cross-sectional view illustrating a state where the slide member of the camera connector constituting the endoscope illustrated in FIG. 1 is at a distal end position.

FIG. 8A is a perspective view illustrating a state where a distal end of the camera constituting the endoscope illustrated in FIG. 1 is located at a first position on a proximal end side from a distal end surface of the distal end tip.

FIG. 8B is a perspective view illustrating a state where the distal end of the camera constituting the endoscope illustrated in FIG. 1 is located at a second position on a distal end side from the distal end surface of the distal end tip.

FIG. 9 is a flow chart schematically illustrating a flow path of a drug solution (passage of forceps), a flow path for water supply, and a flow path for suction in the endoscope illustrated in FIG. 1.

FIG. 10A is an explanatory diagram illustrating a position of an operation lever when a valve mechanism is in an “open” state.

FIG. 10B is an explanatory diagram illustrating a position of the operation lever when the valve mechanism is in a “closed” state.

FIG. 11A is an explanatory diagram illustrating an operation state of the valve mechanism of the endoscope illustrated in FIG. 1.

FIG. 11B is an explanatory diagram illustrating an operation state of the valve mechanism of the endoscope illustrated in FIG. 1.

FIG. 11C is an explanatory diagram illustrating an operation state of the valve mechanism of the endoscope illustrated in FIG. 1.

FIG. 12 is a perspective view of a pressing member constituting the valve mechanism.

FIG. 13 is a perspective view of a tube retaining portion constituting the valve mechanism.

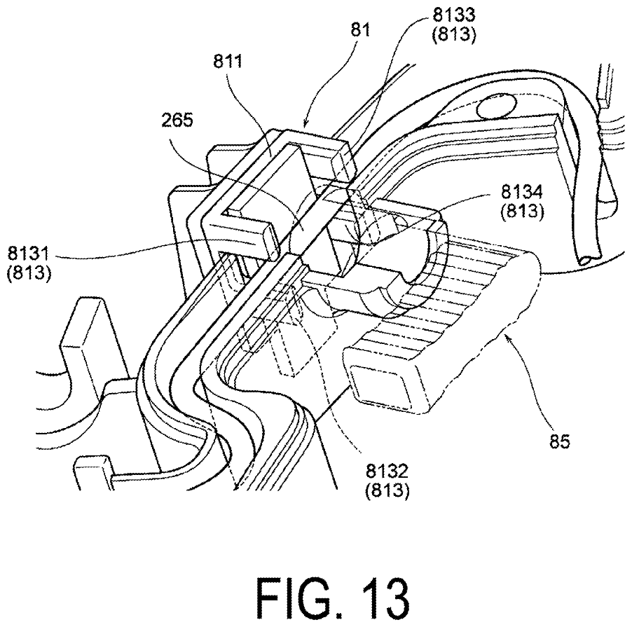

### DESCRIPTION OF EMBODIMENTS

### Embodiments

An embodiment of the disclosure will be described.

An endoscope 100 of this embodiment is inserted into a forceps lumen of a side-view endoscope such as a duodenoscope and is used for diagnostic treatment of disease in a biliary duct or a pancreatic duct.

The endoscope 100 includes a shaft 10 made of resin, including a distal end flexible portion 10A, formed with a camera channel 13, a forceps channel 17 also serving as a suction channel, and two water channels 141 and 142, and formed with four wire lumens 151, 152, 153, and 154;
a handle 20 disposed on a proximal end side of the shaft 10, including a rotational operation portion (an operation knob 251 and an operation knob 252), and formed with a camera port 23 communicating with the camera channel 13, a forceps port 27 communicating with the forceps channel 17, a water port 24 communicating with the water channels 141 and 142 and a suction port 26;
a distal end tip 30 made of resin, disposed on a distal end side of the shaft 10, having an outer diameter equal to that of the shaft 10, and formed with a camera channel 33, water channels 341 and 342, and a forceps channel 37 each opening at a distal end surface 35 of the distal end tip 30 and respectively in communication with the camera channel 13, the water channels 141 and 142, and the forceps channel 17 of the shaft 10;
an intermediate member 40 made of metal, disposed between the shaft 10 and the distal end tip 30, having a disk shape with an outer diameter equal to that of the shaft 10, and including a main through-hole 41 that is formed to ensure communication paths 43, 441, 442, and 47 between the camera channel 13, the water channels 141 and 142, and the forceps channel 17 of the shaft 10 and the camera channel 33, the water channels 341 and 342, and the forceps channel 37 of the distal end tip 30, respectively, and surround all of the communication paths 43, 441, 442, and 47, and four sub-through-holes 421, 422, 423, and 424 that are formed to correspond to the positions at which the wire lumens 151, 152, 153, and 154 of the shaft 10 are formed;
four operation wires 51, 52, 53, and 54 respectively including distal end large-diameter portions 511, 521, 531, and 541 that are embedded in the distal end tip 30 and have diameters larger than diameters of the sub-through-holes 421, 422, 423, and 424 of the intermediate member 40, respectively passing through the sub-through-holes 421, 422, 423, and 424, respectively extending in the wire lumens 151, 152, 153, and 154 of the shaft 10, and including respective tail ends that are fixed to the rotational operation portion (the operation knob 25 or the operation knob 26) of the handle 20 and thereby can be pulled;
a camera 60 including a camera head 61 equipped with a CMOS image sensor 611 (image capturing element) and a cable tube 62, incorporating a plurality of optical fibers 65, and removably disposed with respect to the shaft 10 and the handle 20 by being inserted inside the camera channel 13 and the handle 20;
a suction tube 265 having a proximal end portion connected to a suction pump 96, inserted inside the handle 20 from the suction port 26, extending inside the handle 20, and having a distal end portion joining a communication path between the forceps channel 17 and the forceps port 27; and
a water tube 245, which is a tube being inserted inside the handle 20 from the water port 24 and extending inside the handle 20, having a proximal end portion connected to a water tank 94, and having a lumen communicating with the water channels 141 and 142.

The handle 20 includes a valve mechanism 80 that opens and closes a suction flow path of the suction tube 265.

The endoscope 100 includes the shaft 10 to be inserted into a body, the handle 20 disposed on a proximal end side of the shaft 10, the suction tube 265, the water tube 245, the distal end tip 30 disposed on a distal end side of the shaft 10, the intermediate member 40 disposed between the shaft 10 and the distal end tip 30, the operation wires 51, 52, 53, and 54, the camera 60, and the valve mechanism 80.

### Shaft 10

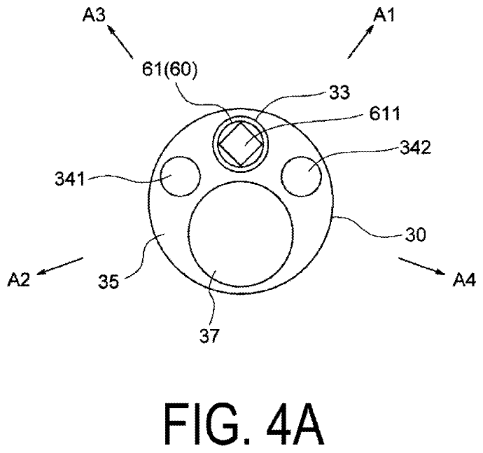

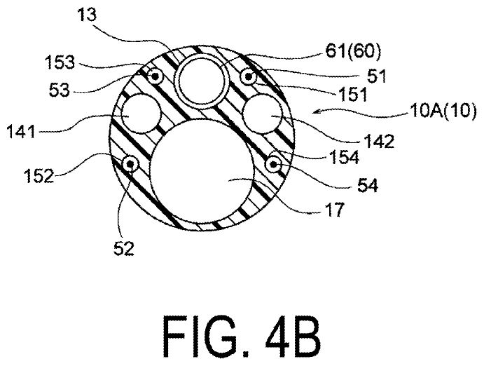

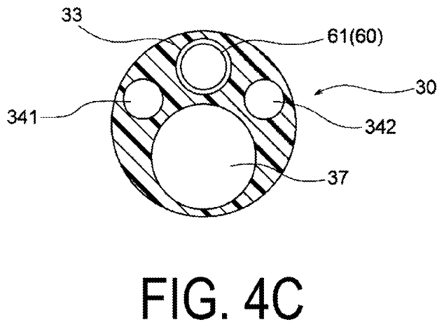

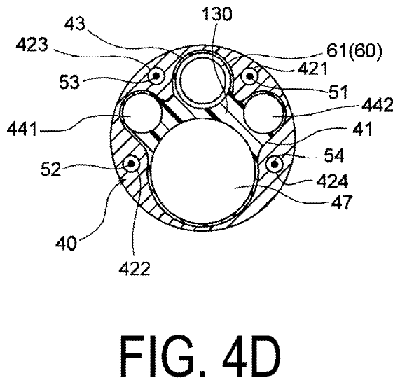

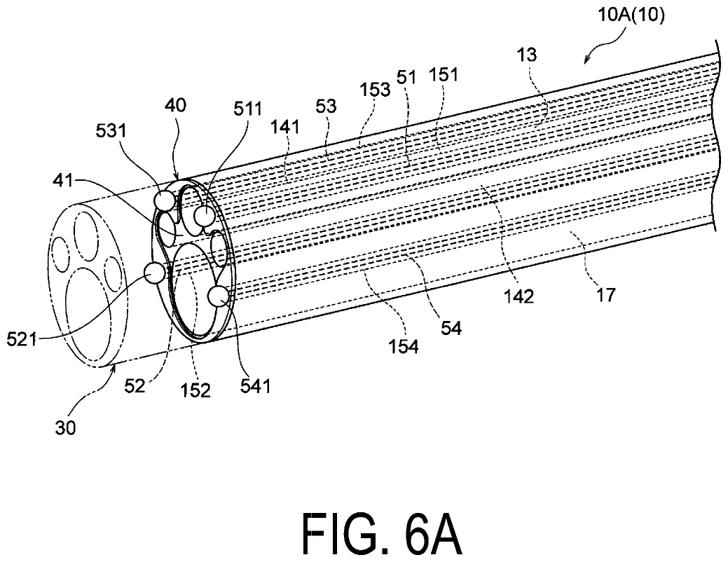

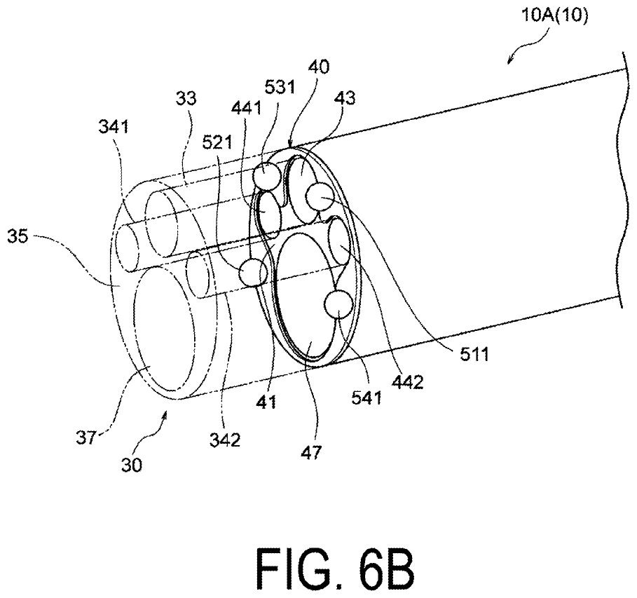

As illustrated in FIGS. 4B and 6A, the camera channel 13, the water channels 141 and 142, and the forceps channel 17 are formed in the shaft 10 constituting the endoscope 100.

Also, the wire lumens 151, 152, 153, and 154 that are insertion paths for the operation wires 51, 52, 53, and 54, respectively, are formed in the shaft 10.

The length (effective length) of the shaft 10 is preferably 200 to 4800 mm and is 1900 mm as one preferred example.

The shaft 10 includes the distal end flexible portion 10A.

Here, the “distal end flexible portion” refers to a distal end portion of the shaft, which can be warped (bent) by pulling the tail end of the operation wire.

The length of the distal end flexible portion 10A is preferably 5 to 200 mm and is 20 mm as one preferred example.

The outer diameter of the shaft 10 is typically 2.8 to 4.1 mm, preferably 3.2 to 3.7 mm, and is 3.6 mm as one preferred example.

The shaft 10 having such a small diameter can be inserted into a biliary duct or a pancreatic duct to perform diagnostic treatment in the duct.

The diameter of the camera channel 13 is typically 0.75 to 1.2 mm, preferably 0.95 to 1.1 mm, and is 1.05 mm as one preferred example.

A live body forceps is inserted into the forceps channel 17, and a drug solution can flow through the forceps channel 17.

In addition, the forceps channel 17 also serves as a suction channel into which body fluid or the like around the distal end portion of the shaft 10 can flow from the forceps opening and through which the body fluid flow.

The diameter of the forceps channel 17 is preferably 1.8 to 3.1 mm, more preferably 1.9 to 2.1 mm, and is 2.0 mm as one preferred example.

When the diameter of the forceps channel 17 is equal to or larger than 1.8 mm, a general-purpose live body forceps having an outer diameter of about 1.75 mm, which cannot be inserted by a conventional endoscope, can be inserted, and when the diameter of the forceps channel 17 is equal to or larger than 1.9 mm, a general-purpose live body forceps having an outer diameter of about 1.85 mm can also be inserted.

By using such a general-purpose live body forceps, a sufficient amount of tissue can be collected at a time of biopsy, and favorable clinical results can be obtained.

Furthermore, such a general-purpose live body forceps is remarkably inexpensive as compared with a forceps having an outer diameter of 4 Fr (1.33 mm) or less that can be inserted by a conventional endoscope.

The diameter of the water channels 141 and 142 is preferably 0.4 to 1.0 mm and is 0.75 mm as one preferred example.

The diameter of the wire lumens 151, 152, 153, and 154 is preferably 0.2 to 0.5 mm, and is 0.33 mm as one preferred example.

The shaft 10 is made of resin.

Examples of the resin material forming the shaft 10 can include nylon resin, polyether block amide (PEBAX) resin, polyurethane resin, polyolefin resin, and the like, and of these resins, PEBAX resin and polyurethane resin are preferable.

The hardness (Shore D hardness) of the constituent resin of the shaft 10 is preferably 90 D or less, and when one preferred example is given, the hardness of the resin forming the distal end flexible portion 10A is 25 D, and the hardness of the resin forming a portion other than the distal end flexible portion 10A is 30 D.

### Handle 20

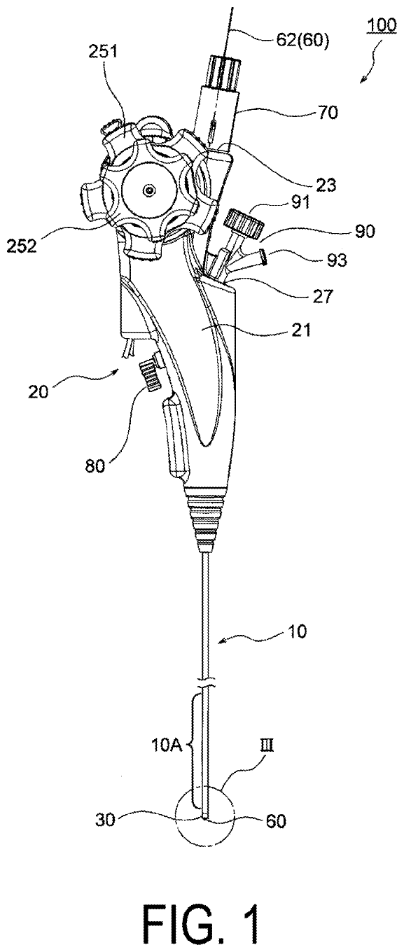

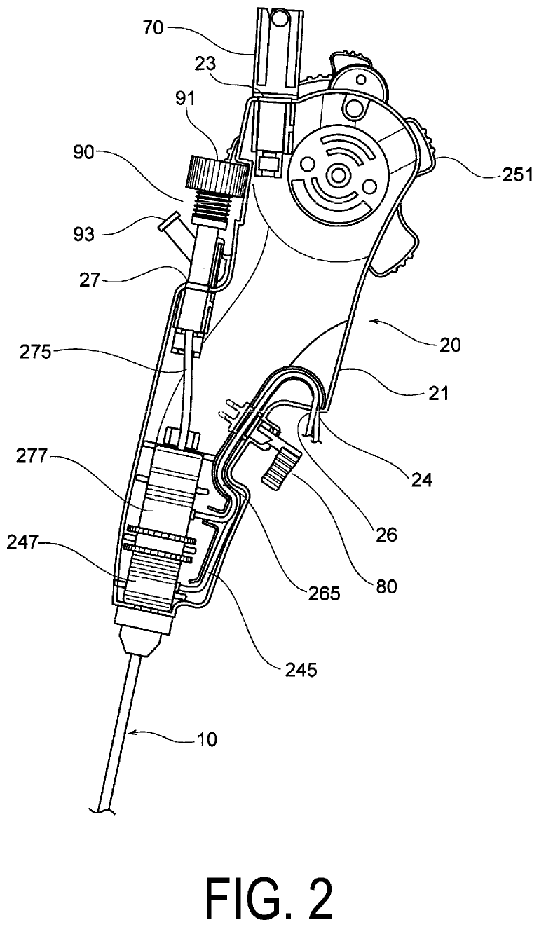

As illustrated in FIGS. 1 and 2, the handle 20 is disposed on a proximal end side of the shaft 10. The handle 20 constituting the endoscope 100 includes a grip 21 and the two operation knobs 251 and 252 as a rotational operation portion.

The handle 20 is provided with the camera port 23 communicating with the camera channel 13, the forceps port 27 communicating with the forceps channel 17, the water port 24 communicating with the water channels 141 and 142, and the suction port 26 communicating with the forceps channel 17.

A camera connector 70 is attached to the camera port 23.

A Y connector 90 including a forceps insertion port 91 and a drug solution injection port 93 is attached to the forceps port 27.

As illustrated in FIG. 2, a first chamber 277 and a second chamber 247 are disposed inside the handle 20.

The first chamber 277 communicates with the forceps channel 17 of shaft 10.

The second chamber 247 communicates with the water channels 141 and 142 of the shaft 10.

In FIG. 2, a reference sign 275 denotes a forceps tube, and the forceps tube 275 communicates between the forceps port 27 and the first chamber 277.

Accordingly, the forceps port 27 of the handle 20 communicates with the forceps channel 17 of the shaft 10 via the forceps tube 275 and the first chamber 277.

In FIG. 2, a reference sign 245 denotes a water tube whose proximal end portion is connected to a water tank (not illustrated). The water tube 245 is inserted inside the handle 20 from the water port 24 and extends inside the handle 20, and the distal end portion of the water tube 245 is connected to the second chamber 247. Accordingly, the lumen of the water tube 245 communicates with the water channels 141 and 142.

In FIG. 2, a reference sign 265 denotes a suction tube whose proximal end portion is connected to a suction pump (not illustrated). The suction tube 265 is inserted inside the handle 20 from the suction port 26 and extends inside the handle 20, and the distal end portion of the suction tube 265 is connected to the first chamber 277. Accordingly, the lumen of the suction tube 265 communicates with the forceps channel 17 and the forceps port 27.

In FIG. 2, a reference sign 80 denotes a valve mechanism that opens and closes a suction flow path of the suction tube 265, which will be described in detail below.

### Distal End Tip 30

The distal end tip 30 is disposed on the distal end side of the shaft 10.

As illustrated in FIGS. 4A, 4C, and 6B, the camera channel 33, the water channels 341 and 342, and the forceps channel 37 are formed in the distal end tip 30 constituting the endoscope 100.

The camera channel 33 communicates with the camera channel 13 of the shaft 10 via the communication path 43. The diameter of the camera channel 33 is equal to the diameter of the camera channel 13 communicating with the camera channel 33.

The water channels 341 and 342 respectively communicate with the water channels 141 and 142 of the shaft 10 via the communication paths 441 and 442. The diameter of the water channels 341 and 342 is equal to the diameter of the water channels 141 and 142 respectively communicating with the water channels 341 and 342.

The forceps channel 37 communicates with the forceps channel 17 of shaft 10 via the communication path 47. The diameter of the forceps channel 37 is equal to the diameter of the forceps channel 17 communicating with the forceps channel 37.

The length of the distal end tip 30 is preferably 1 to 30 mm and is 3 mm as one preferred example.

The outer diameter of the distal end tip 30 is equal to the outer diameter of the shaft 10.

The distal end tip 30 is made of resin.

Examples of the resin material forming the distal end tip 30 can include resins similar to those illustrated as the resins forming the shaft 10, and out of the resins, PEBAX resin and polyurethane resin are preferable.

The distal end tip 30 is made of a resin material having low hardness so as not to damage body tissue. The hardness (Shore D hardness) of the constituent resin of the distal end tip 30 is preferably 72 D or less and is 25 D as one preferred example.

### Intermediate Member 40

The intermediate member 40 formed in a disk shape is disposed between the shaft 10 and the distal end tip 30.

The intermediate member 40 constituting the endoscope 100 is a member for fixing a distal end of each of the operation wires 51, 52, 53 and 54 to a distal end of the shaft 10 (for suppressing removal of the tail end during pulling operation).

As illustrated in FIGS. 4D and 6B, one main through-hole 41 surrounding all of the communication path 43, the communication paths 441 and 442, and the communication path 47 is formed in the intermediate member 40.

Here, the communication path 43 is a path (camera channel) defined and formed by a constituent resin 130 of the shaft 10 and/or the distal end tip 30 in order to allow the camera channel 13 of the shaft 10 to communicate with the camera channel 33 of the distal end tip 30. The diameter of the communication path 43 is equal to the diameters of the camera channel 13 and the camera channel 33.

The communication paths 441 and 442 are paths (water channels) defined and formed by the constituent resin 130 of the shaft 10 and/or the distal end tip 30 in order to allow the water channels 141 and 142 of the shaft 10 to respectively communicate with the water channels 341 and 342 of the distal end tip 30. The diameter of the communication paths 441 and 442 is equal to the diameters of the water channels 141 and 142 and the water channels 341 and 342.

The communication path 47 is a path (forceps channel) defined and formed by the constituent resin 130 of the shaft 10 and/or the distal end tip 30 to allow the forceps channel 17 of the shaft 10 to communicate with the forceps channel 37 of the distal end tip 30. The diameter of the communication path 47 is equal to the diameters of the forceps channel 17 and the forceps channel 37.

As illustrated in FIG. 4D, four sub-through-holes 421, 422, 423 and 424 are formed in the intermediate member 40 so as to correspond to the positions at which the wire lumens 151, 152, 153, and 154 of the shaft 10 are formed. The sub-through-holes 421, 422, 423, and 424 are respectively insertion paths for the operation wires 51, 52, 53, and 54.

The sub-through-holes 421, 422, 423, and 424 are circular holes, and the diameter of each of the sub-through-holes is larger than the diameter of the corresponding one of operation wires 51, 52, 53, and 54 and is adjusted to be smaller than the diameter of the corresponding one of distal end large-diameter portions 511, 521, 531, and 541 to restrict insertion thereof.

The diameter of the sub-through-hole is preferably 0.13 to 2.5 mm and is 0.35 mm as one preferred example.

The thickness of the intermediate member 40 is preferably 0.05 to 3 mm and is 0.15 mm as one preferred example.

When the thickness of the intermediate member 40 is excessively small, the intermediate member 40 may be damaged when receiving a mechanical shock associated with pulling of the operation wires 51, 52, 53, and 54.

On the other hand, when the thickness is excessively large, the intermediate member 40 itself is less likely to warp, and thus the distal end flexible portion 10A may be difficult to warp.

The outer diameter of the intermediate member 40 is equal to the outer diameters of the shaft 10 and the distal end tip 30, and thus the outer circumferential surface of the shaft 10, the outer circumferential surface of the intermediate member 40, and the outer circumferential surface of the distal end tip 30 are flush with one another. Accordingly, the intermediate member 40 does not protrude from between the shaft 10 and the distal end tip 30 and the edge thereof is not exposed, and thus body tissue and the like are not damaged by such an edge.

The intermediate member 40 is made of metal or ceramic and is preferably made of metal.

Example of the metal material forming the intermediate member 40 can include stainless copper, platinum, gold, copper, nickel, titanium, tantalum, and the like, and of the metals, stainless copper is preferable.

In the endoscope 100 of the present embodiment, the shaft 10 and the distal end tip 30 are directly bonded (the constituent resins of the shaft 10 and the distal end tip 30 are welded) in a region on the inner side of the main through-hole 41 of the intermediate member 40 and on the outer side of each of the communication paths 43, 441, 442, and 47 (a region surrounded by the main through-hole 41 excluding the communication paths). Accordingly, the intermediate member 40 is also secured by the resin present on the inner side of the main through-hole 41, and the intermediate member 40 is firmly secured to the shaft 10 and the distal end tip 30.

Also, the shaft 10 and distal end tip 30 are directly bonded, although partially, and thus the fixing strength of the distal end tip 30 with respect to the shaft 10 is also sufficiently increased.

Here, the area of the region in which the constituent resins of the shaft 10 and the distal end tip 30 are directly bonded is (S) and the cross-sectional area of the shaft 10 is (S₀), the value of (S)/(S₀) is preferably 0.1 or greater, and is more preferably 0.3 to 0.6.

### Operation Wire 51, 52, 53, and 54

As illustrated in FIGS. 4B and 6A, the operation wires 51, 52, 53, and 54 extend respectively in the wire lumens 151, 152, 153, and 154 of the shaft 10.

As illustrated in FIGS. 6A and 6B, the distal ends of the operation wires 51, 52, 53, and 54 are the distal end large-diameter portions 511, 521, 531, and 541, respectively.

The distal end large-diameter portions 511, 521, 531, and 541 respectively have spherical or partially spherical shapes having a diameter larger than the diameter of the sub-through-holes 421, 422, 423, and 424 of the intermediate member 40, and cannot pass through the sub-through-holes 421, 422, 423, and 424.

The diameter of the distal end large-diameter portions 511, 521, 531, and 541 is preferably 0.2 to 3.5 mm, and is 0.4 mm as one preferred example.

The diameter of the operation wires 51, 52, 53, and 54 (a portion other than the distal end large-diameter portion) is preferably 0.1 to 2.0 mm and is 0.25 mm as one preferred example.

The operation wires 51 and 52 are held in a state where each of the distal end large-diameter portions 511 and 521 is embedded in the distal end tip 30, and portions of the wires, which are located on the proximal end side of the distal end large-diameter portions 511 and 521 respectively pass through the sub-through-holes 421 and 422 of the intermediate member 40 and extend in the wire lumens 151 and 152 of the shaft 10. Each of the proximal ends of the operation wires 51 and 52 is fixed to the operation knob 25 of the handle 20.

By rotating the operation knob 25 in one direction and pulling the proximal end of the operation wire 51, the operation wire 51 moves the wire lumen 151 in a proximal end direction. At this time, the distal end large-diameter portion 511 gets caught on the sub-through-hole 421 of the intermediate member 40 to be restricted from moving in the proximal end direction, and thus the distal end flexible portion 10A of the shaft 10 warps in the direction indicated by an arrow A1 in FIG. 4A, and the distal end of the endoscope 100 (the distal end tip 30) deflects in the same direction.

By rotating the operation knob 25 in the other direction and pulling the proximal end of the operation wire 52, the operation wire 52 moves the wire lumen 152 in the proximal end direction. At this time, the distal end large-diameter portion 521 gets caught on the sub-through-hole 422 of the intermediate member 40 to be restricted from moving in the proximal end direction, and thus the distal end flexible portion 10A of the shaft 10 warps in the direction indicated by an arrow A2 in FIG. 4A, and the distal end of the endoscope 100 (the distal end tip 30) deflects in the same direction.

The operation wires 53 and 54 are held in a state where each of the distal end large-diameter portions 531 and 541 is embedded in the distal end tip 30, and portions of the wires, which are located on the proximal end side of the distal end large-diameter portions 531 and 541 respectively pass through the sub-through-holes 423 and 424 of the intermediate member 40 and extend in the wire lumens 153 and 154 of the shaft 10. Each of the proximal ends of the operation wires 53 and 54 is fixed to the operation knob 26 of the handle 20.

By rotating the operation knob 26 in one direction and pulling the proximal end of the operation wire 53, the operation wire 53 moves the wire lumen 153 in the proximal end direction. At this time, the distal end large-diameter portion 531 gets caught on the sub-through-hole 423 of the intermediate member 40 to be restricted from moving in the proximal end direction, and thus the distal end flexible portion 10A of the shaft 10 warps in the direction indicated by an arrow A3 in FIG. 4A, and the distal end of the endoscope 100 (the distal end tip 30) deflects in the same direction.

By rotating the operation knob 26 in the other direction and pulling the proximal end of the operation wire 54, the operation wire 54 moves the wire lumen 154 in the proximal end direction. At this time, the distal end large-diameter portion 541 gets caught on the sub-through-hole 424 of the intermediate member 40 to be restricted from moving in the proximal end direction, and thus the distal end flexible portion 10A of the shaft 10 warps in the direction indicated by an arrow A4 in FIG. 4A, and the distal end of the endoscope 100 (the distal end tip 30) deflects in the same direction.

When the tail ends of the operation wires 51, 52, 53, and 54 are pulled as described above, the distal end large-diameter portions 511, 521, 531, and 541 respectively get caught on the sub-through-holes 421, 422, 423, and 424 formed in the intermediate member 40, and the distal ends of the operation wires 51, 52, 53, and 54 are fixed to the distal end of the shaft 10 (are suppressed from being removed), and thus the distal end flexible portion 10A of the shaft 10 can be warped in the desired direction (the directions indicated by arrows A1 to A4).

Without the intermediate member disposed between the shaft and the distal end tip, when the proximal end of the operation wire is pulled, the distal end of the operation wire (the distal end large-diameter portion) cannot be sufficiently fixed with respect to the distal end of the shaft (suppressed from being removed) and the distal end large-diameter portion moves in the proximal end direction while pushing and expanding the wire lumen. In this case, the distal end flexible portion cannot be warped.

The constituent material of the operation wires 51, 52, 53, and 54 is not particularly limited, and the same material as the constituent material of operation wires used in a conventionally known medical device that can perform distal end deflection operation can be used.

### Camera 60

The camera 60 constituting the endoscope 100 includes the camera head 61 equipped with the CMOS image sensor 611, and the cable tube 62 incorporating a transmission cable of the CMOS image sensor 611.

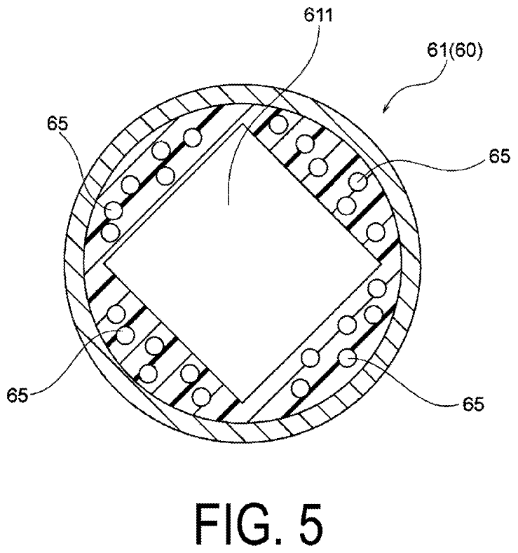

As illustrated in FIG. 5, the camera 60 includes the plurality of (24 in the example illustrated in the drawing) optical fibers 65 surrounding the CMOS image sensor 611.

Accordingly, it is not necessary to separately form a channel for disposing the optical fibers, and it is possible to sufficiently reduce the diameter of the shaft 10 and the size of a device.

In addition, because the camera 60 constituting the endoscope 100 is disposed so as to be removable with respect to the shaft 10 (not fixed to the shaft 10), when the distal end flexible portion 10A of the shaft 10 is bent, the camera 60 moves in the camera channel 13 in the axial direction, and thus a load on the optical fibers 65 located inside the camera 60 can be reduced.

The outer diameter of the camera head 61 is preferably 0.7 to 1.0 mm and is 1.0 mm as one preferred example. The outer diameter of the cable tube 62 is substantially equal to the outer diameter of the camera head 61.

The camera 60 is disposed in the camera channels (the camera channel 13 and the camera channel 33) of the shaft 10 and the distal end tip 30. A proximal end portion of the cable tube 62 extends out from the camera port 23 of the handle 20 to the outside, and a proximal end of the cable tube is connected to a control device.

The camera connector 70 is attached to the cable tube 62 of the camera 60.

The camera connector 70 is mounted to the camera port 23 of the handle 20 when the camera 60 is properly disposed in the camera channel 13 and the camera channel 33.

In other words, by mounting the camera connector 70 to the camera port 23, the camera 60 is properly disposed in the camera channel 13 and the camera channel 33.

The mounting position of the camera connector 70 is 300 to 5000 mm from a distal end of the camera 60 and is 2100 mm from the distal end of the camera 60 as one preferred example.

In the endoscope 100 of the present embodiment, the camera 60 is separable from the handle 20 and the shaft 10.

In other words, the camera connector 70 is removed from the camera port 23, and then the camera 60 disposed in the camera channel 13 and the camera channel 33 can be removed from the camera port 23 of the handle 20 along with the camera connector 70.

Also, once separated, the camera 60 can be reincorporated as a component of the endoscope 100 by inserting, with the camera head 61 first, the camera 60 inside the handle 20 and the camera channel 13 of the shaft 10 from the camera port 23 of the handle 20 and mounting the camera connector 70 to the camera port 23.

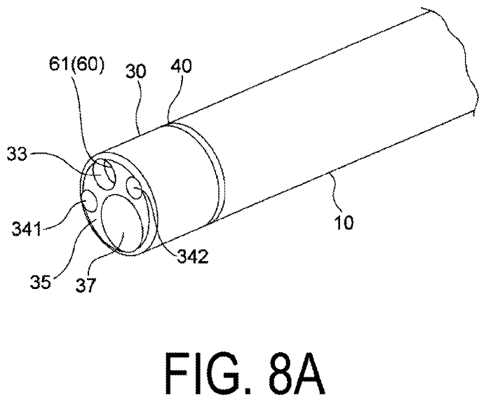

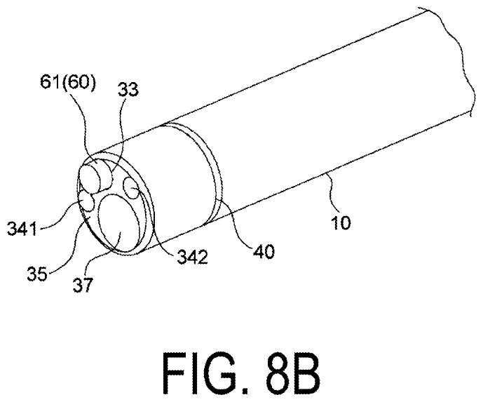

The camera connector 70 includes a camera position adjustment mechanism that causes the camera 60 to reciprocate with respect to the camera channel 13 and the camera channel 33 such that a distal end of the camera 60 disposed in the camera channel 13 and the camera channel 33 shifts between a first position (a distal end position of the camera 60 as illustrated in FIG. 8A) located on the proximal end side from the distal end surface 35 of the distal end tip 30 in which the camera channel 33 opens and a second position (a distal end position of the camera 60 as illustrated in FIG. 8B) located on the distal end side from the distal end surface 35 when mounted to the camera port 23.

Here, the distance from the first position to the second position (the movement distance of the distal end of the camera 60 by the position adjustment mechanism) is preferably 2 to 100 mm and is 30 mm as one preferred example.

In addition, the distance from the distal end surface 35 of the distal end tip 30 to the first position is preferably 1.5 to 20 mm, and the distance from the distal end surface 35 to the second position is preferably 0.5 to 80 mm.

In the endoscope 100 of the present embodiment, the camera position adjustment mechanism included in the camera connector 70 is a mechanism that reciprocates the camera 60 by using a feed screw.

Specifically, the mechanism includes a connector case 71 mounted to the camera port 23, formed with a guide groove (not illustrated) extending along the axial direction on an inner circumferential surface, and formed with a guide hole 713 extending along the axial direction in a peripheral wall;
a slide member 72 slidable with respect to the connector case 71 and including a shaft portion 721 extending inside the connector case 71 and partially extending out to the proximal end side of the connector case 71, having a proximal end portion on which an external threaded portion 722 is formed, and into which the cable tube 62 of the camera 60 is inserted and adhesively fixed and a guide portion 723 integrally formed with the shaft portion 721 to surround a distal end portion of the shaft portion 721, formed with a projected portion (not illustrated) to be guided by the guide groove of the connector case 71 on an outer circumferential surface, and formed with a protruded portion 725 to be guided by the guide hole 713 on the outer circumferential side; and
a rotation knob 73 located on the proximal end side of the connector case 71 to be restricted from moving in the axial direction and including an internal threaded portion 731 that screws with the external threaded portion 722 of the shaft portion 721 of the slide member 72.

By rotating the rotation knob 73 in one direction to slide the slide member 72 from a proximal end position to a distal end position, the distal end of the camera 60 is moved from the first position (the distal end position of the camera 60 as illustrated in FIG. 8A) to the second position (the distal end position of the camera 60 as illustrated in FIG. 8B). By rotating the rotation knob 73 in the other direction to slide the slide member 72 from the distal end position to the proximal end position, the distal end of the camera 60 is moved from the second position to the first position.

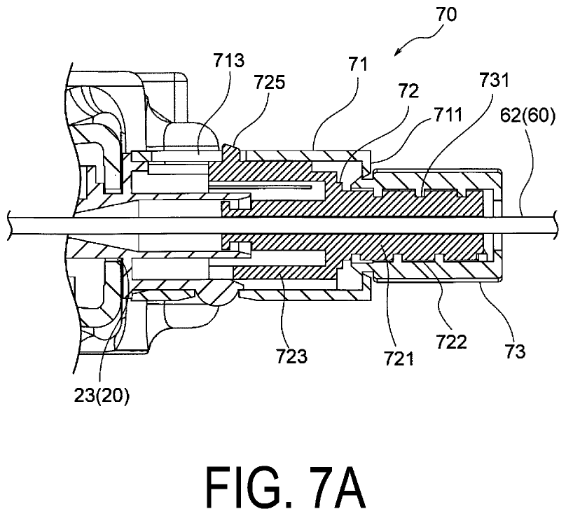

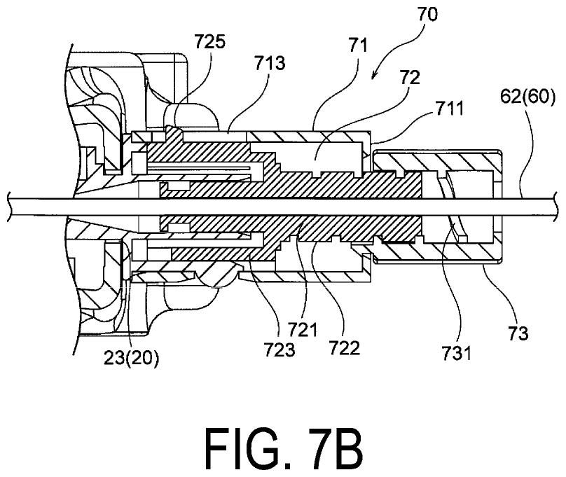

Here, the “proximal end position” is a position from which the slide member 72 cannot move further toward the proximal end side as illustrated in FIG. 7A, and the “distal end position” is a position from which the slide member 72 cannot move further toward the distal end side as illustrated in FIG. 7B.

The camera position adjustment mechanism includes the connector case 71, the slide member 72, and the rotation knob 73.

The connector case 71 is a constituent member of the camera connector 70 mounted to the camera port 23 via a port-side connector to be described below and includes a tubular body having an arch-shaped portion.

The guide groove extending along the axial direction is formed on the inner circumferential surface of the connector case 71, and the guide hole 713 extending along the axial direction is formed in the peripheral wall of the arch-shaped portion.

The slide member 72 includes the shaft portion 721 and the guide portion 723 integrally formed with the shaft portion 721 to surround the distal end portion of the shaft portion 721.

The shaft portion 721 of the slide member 72 extends inside the connector case 71, and a part of the shaft portion 721 extends out from an opening formed in a proximal end surface 711 of the connector case 71 to the proximal end side.

The external threaded portion 722 is formed on the proximal end portion of the shaft portion 721.

As illustrated in FIGS. 7A and 7B, the cable tube 62 of the camera 60 is adhesively fixed inside the shaft portion 721 while being inserted thereinto.

The guide portion 723 of the slide member 72 has an arch-shaped portion along with the shape of the connector case 71 and is integrally formed with the shaft portion 721 to surround the distal end portion of the shaft portion 721.

The projected portion to be guided by the guide groove of the connector case 71 is formed on the outer circumferential surface of the guide portion 723.

In addition, the protruded portion 725 to be guided by the guide hole 713 of the connector case 71 is formed on the outer circumferential side of the arch-shaped portion of the guide portion 723.

The rotation knob 73 is disposed on the proximal end side of the connector case 71.

The internal threaded portion 731 that screws with the external threaded portion 722 of the shaft portion 721 of the slide member 72 is formed on the inner circumferential side of the rotation knob 73.

The rotation knob 73 is restricted from moving in the axial direction with respect to the connector case 71. By rotating the rotation knob 73, the slide member 72 slides with respect to the connector case 71.

In addition, by rotating the rotation knob 73 to slide the slide member 72, the cable tube 62 adhesively fixed inside the shaft portion 721 also moves in the axial direction with respect to the connector case 71.

With the camera position adjustment mechanism configured as described above, the distal end of the camera 60 can be moved from the first position (the distal end position of the camera 60 as illustrated in FIG. 8A) to the second position (the distal end position of the camera 60 as illustrated in FIG. 8B) by rotating the rotation knob 73 in one direction to slide the slide member 72 from the proximal end position (the position illustrated in FIG. 7A) to the distal end position (the position illustrated in FIG. 7B), and the distal end of the camera 60 can be moved from the second position to the first position by rotating the rotation knob 73 in the other direction to slide the slide member 72 from the distal end position to the proximal end position.

### Valve Mechanism 80

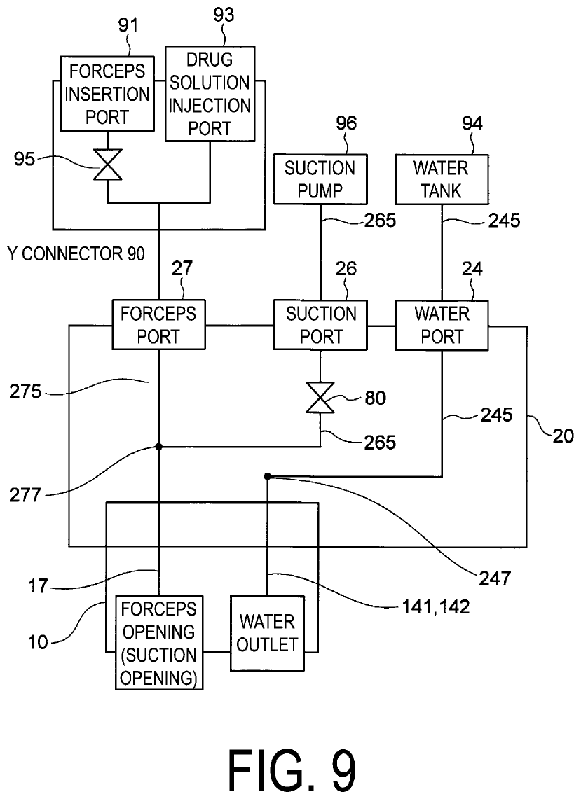

FIG. 9 is a flow chart schematically illustrating a flow path of a drug solution (forceps passage), a flow path for water supply, and a suction flow path for suction in the endoscope 100 of the present embodiment.

The handle 20 is provided with the forceps port 27, the water port 24, and the suction port 26.

The Y connector 90 including the forceps insertion port 91 and the drug solution injection port 93 is connected to the forceps port 27 of the handle 20. A reference sign 95 denotes an opening/closing valve of the forceps insertion port 91.

By inserting a forceps from the forceps insertion port 91 in a state where the opening/closing valve 95 of the Y connector 90 is open, the forceps can be inserted inside the handle 20 (the forceps tube 275 and the first chamber 277) and the forceps channel 17 of the shaft 10, and the distal end portion of the forceps can be extended out from the forceps opening (the distal end opening of the forceps channel).

In addition, by injecting a drug solution from the drug solution injection port 93 in a state where the opening/closing valve 95 of the Y connector 90 is closed, the drug solution can flow inside the handle 20 (the forceps tube 275 and the first chamber 277) and the forceps channel 17 of the shaft 10 and can flow out (can be administered) from the forceps opening.

The water tube 245 having a proximal end portion connected to the water tank 94 is inserted inside the handle 20 from the water port 24 and extends inside the handle 20, and a lumen of the water tube 245 communicates with the water channels 141 and 142 of the shaft 10 via the second chamber 247. Accordingly, the water in the water tank 94 can flow out from the water outlet.

The suction tube 265 having a proximal end portion connected to the suction pump 96 is inserted inside the handle 20 from the suction port 26 and extends inside the handle 20, and the distal end portion of the suction tube 265 is connected to the first chamber 277, thereby joining the communication path between the forceps channel 17 and the forceps port 27. Accordingly, by operating the suction pump 96, a body fluid or the like of a patient can be sucked from the forceps opening (suction opening) and discharged to the outside of the body through the forceps channel 17 and the suction tube 265.

The endoscope 100 of the present embodiment is characterized in that the valve mechanism 80 for opening and closing the suction flow path formed by the lumen of the suction tube 265 is provided at the handle 20.

With the valve mechanism 80 provided, when a drug solution is administered (the drug solution is injected from the drug solution injection port 93 of the Y connector 90) while the suction pump 96 is operating, the drug solution flowing through the forceps tube 275 can be suppressed from being sucked into the suction tube 265 by closing the suction flow path of the suction tube 265 by the valve mechanism 80, and the drug solution can flow through the forceps channel 17 and can flow out (can be administered) from the forceps opening.

Then, by opening the suction flow path of the suction tube 265 after the administration of the drug solution, the operation of sucking the body fluid or the like of the patient and discharging the same to the outside of the body ban be resumed immediately.

In addition, because the valve mechanism 80 is provided at the handle 20, the opening and closing operation of the suction flow path of the suction tube 265 can be easily performed while the handle 20 is being gripped.

When such a valve mechanism is provided outside the handle (at the suction tube between the suction port and the suction pump), the handle is gripped (operated) by one hand while the opening and closing operation is performed by the other hand, which makes the opening and closing operation extremely difficult.

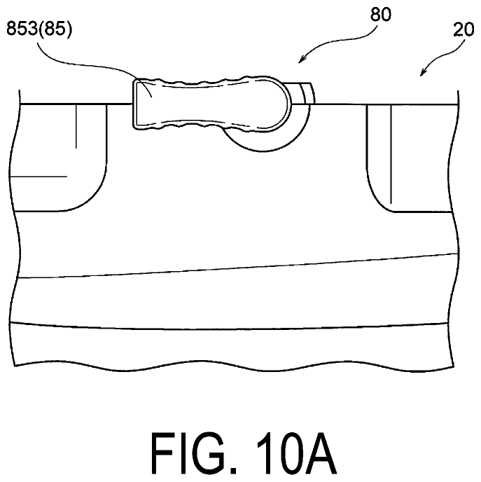

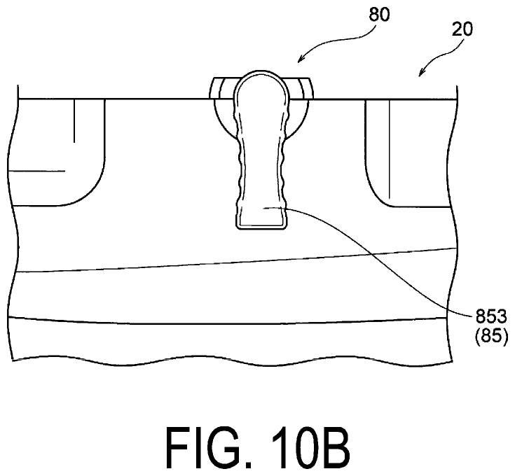

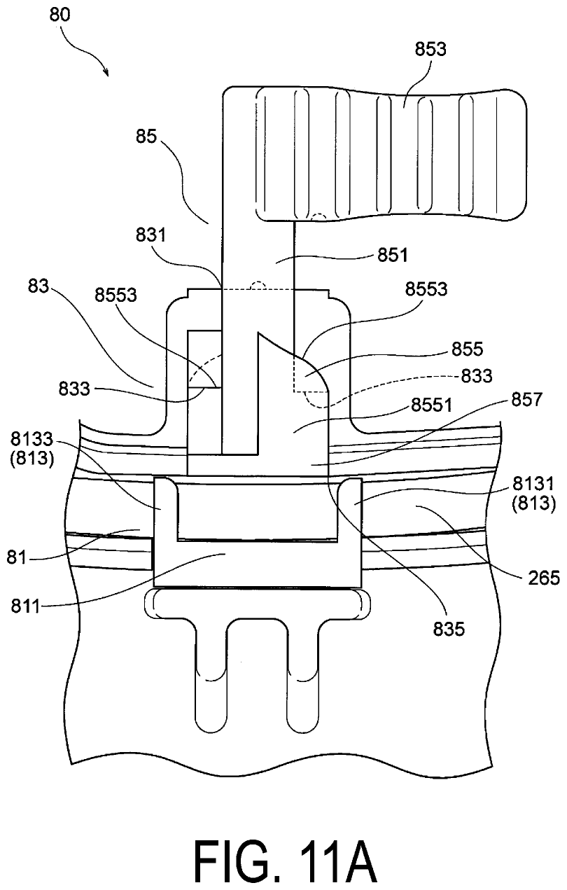

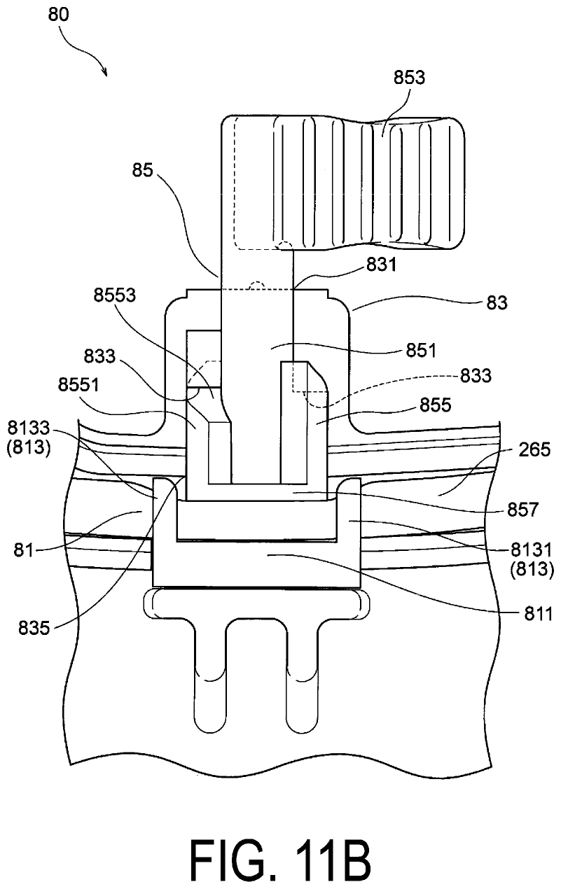

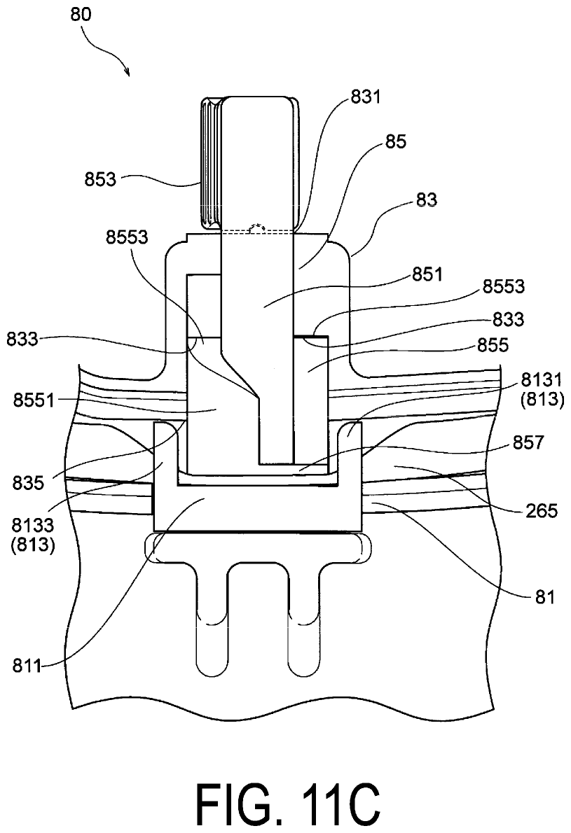

FIGS. 10A, 10B, and 11A to 11C illustrate the valve mechanism 80 in the endoscope 100 of the present embodiment.

FIGS. 10A and 11A illustrate an “open” state in which the suction flow path of the suction tube 265 is open, FIGS. 10B and 11C illustrate a “closed” state in which the suction flow path of the suction tube 265 is closed, and FIG. 11B illustrates an intermediate state between “open” and “closed”.

The valve mechanism 80 includes a tube retaining portion 81, a cylinder portion 83, and a pressing member 85.

The tube retaining portion 81 constituting the valve mechanism 80 is disposed inside the handle 20 so as to retain the suction tube 265 (a portion whose outer circumferential surface is to be pressed) extending inside the handle 20 at a predetermined position.

The tube retaining portion 81 includes a flat plate portion 811, a tube gripping portion 813, and a partition wall portions (8132 and 8134).

The flat plate portion 811 has a flat surface facing a distal end surface of the pressing member 85 to be described below.

The tube gripping portion 813 includes protruding plates 8131 to 8134 extending in a direction perpendicular to the flat plate portion 811 on both sides of the flat plate portion 811. The suction tube 265 is inserted between the protruding plate 8131 and the protruding plate 8132 and between the protruding plate 8133 and the protruding plate 8134, whereby the suction tube 265 is retained (sandwiched).

The protruding plate 8132 and the protruding plate 8134 constituting the tube gripping portion 813 form the partition wall portion. The suction tube 265 and the water tube 245 extending in parallel with the suction tube 265 can be spaced apart from each other by the partition wall portion (8132 and 8134), whereby the outer circumferential surface of the water tube 245 can be suppressed from being pressed (the flow path of water is suppressed from being closed).

The cylinder portion 83 constituting the valve mechanism 80 is formed at the handle 20 so as to communicate between the inside and the outside of the handle 20.

The cylinder portion 83 is formed with a proximal end opening 831 through which a shaft portion 851 of the pressing member 85 to be described below is inserted.

The pressing member 85 constituting the valve mechanism 80 moves inside the cylinder portion 83 along a direction of an axis of the cylinder portion 83 while rotating around the axis.

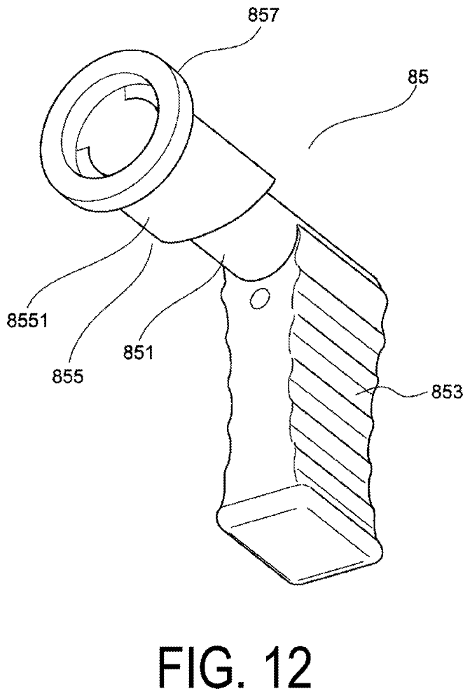

As illustrated in FIG. 12, the pressing member 85 is formed by integrally molding the shaft portion 851, an operation lever 853, a partial outer circumferential portion 855, and a distal end portion 857.

A proximal end portion of the shaft portion 851 extends out from the proximal end opening 831 of the cylinder portion 83 to the outside of the handle 20, and the operation lever 853 for rotating the pressing member 85 is attached to a proximal end portion of the shaft portion 851.

The partial outer circumferential portion 855 of the pressing member 85 is formed so as to cover a part (0° to 90° and 180° to 270°) of the outer circumferential surface of the shaft portion 851 in the circumferential direction and includes an outer circumferential surface 8551 slidable with respect to an inner circumferential surface of the cylinder portion 83 and a proximal end surface 8553 having a helical shape and being slidable with respect to an inner end surface 833 of the cylinder portion 83.

As illustrated in FIG. 12, the distal end portion 857 of the pressing member 85 has an annular shape. With the distal end portion 857 having an annular shape, an area to be pressed against the outer circumferential surface of the suction tube 265 is reduced and the pressing force can be concentrated, and thus the suction flow path of the suction tube 265 can be more reliably closed.

When the shaft portion 851 is rotated by the operation lever 853 from the “open” state illustrated in FIG. 10A (FIG. 11A), the proximal end surface 8553 of the partial outer circumferential portion 855 slides with respect to the inner end surface 833 of the cylinder portion 83. Along with this, as illustrated in FIG. 11B, the pressing member 85 moves along the direction of the axis of the cylinder portion 83 while rotating around the axis, and the distal end portion 857 of the pressing member 85 located (waiting) inside the cylinder portion 83 extends out from the distal end opening 835 of the cylinder portion 83 into the handle 20, whereby the distal end surface of the pressing member 85 presses the outer circumferential surface of the suction tube retained by the tube gripping portion 813.

When the operation lever 853 is rotated to the position illustrated in FIG. 10B, the outer circumferential surface of the suction tube 265 is sufficiently pressed by being sandwiched between the distal end surface of the pressing member 85 and the flat plate portion 811 as illustrated in FIG. 11C, whereby the suction flow path is brought into the “closed” state in which the suction flow path is completely closed.

In the endoscope 100 of the present embodiment, because the handle 20 includes the valve mechanism 80 for opening and closing the suction flow path of the suction tube 265, opening and closing operations on the suction flow path of the suction tube 265 can be easily performed by rotating the operation lever 853 while the handle 20 is being gripped.

Accordingly, when a drug solution is injected from the forceps port 27 of the handle 20 while the suction pump is operating, the valve mechanism 80 can be put into the “closed” state, and the injected drug solution can reliably flow out from the forceps opening at the distal end of the shaft 10.

In addition, the tube gripping portion 813 including the protruding plates 8131 to 8134 can retain the suction tube 265 (a portion whose outer circumferential surface is to be pressed) at a predetermined position.

In addition, the partition wall portion including the protruding plate 8132 and the protruding plate 8134 can suppress the outer circumferential surface of the water tube 245, which extends in parallel with the suction tube 265, from being pressed (the flow path for water supply is suppressed from being closed).

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An endoscope comprising:
a shaft including a forceps channel and a water channel, the forceps channel also serving as a suction channel;
a handle disposed on a proximal end side of the shaft, the handle including a forceps port, a water port, and a suction port, the forceps port communicating with the forceps channel;
a camera disposed inside the shaft and the handle;
a suction tube having a proximal end portion connected to a suction unit, the suction tube being inserted inside the handle from the suction port and extending inside the handle, the suction tube having a distal end portion joining a communication path between the forceps channel and the forceps port; and
a water tube having a proximal end portion connected to a water supply unit, a tube being inserted inside the handle from the water port and extending inside the handle, the water tube having a lumen communicating with the water channel,
wherein the handle includes a valve mechanism configured to open and close a suction flow path of the suction tube.

2. The endoscope according to claim 1, wherein the valve mechanism is put into a “closed” state by closing the suction flow path by pressing an outer circumferential surface of the suction tube.

3. The endoscope according to claim 2,
wherein the valve mechanism includes a tube retaining portion that is disposed inside the handle and retains the suction tube extending inside the handle at a predetermined position, a cylinder portion that is attached to or formed at the handle and communicates between an inside and an outside of the handle, and a pressing member that moves along a direction of an axis of the cylinder portion while rotating around the axis, and
when the valve mechanism is in an “open” state, a distal end portion of the pressing member is located inside the cylinder portion, and when the valve mechanism is in the “closed” state, the distal end portion of the pressing member extends out from a distal end opening of the cylinder portion to an inside of the handle, and a distal end surface of the pressing member presses an outer circumferential surface of the suction tube retained by the tube retaining portion.

4. The endoscope according to claim 3,
wherein the pressing member is formed by integrally molding a shaft portion including a proximal end portion extending out from a proximal end opening of the cylinder portion to an outside of the handle,
an operation lever attached to the proximal end portion of the shaft portion and configured to cause a rotating operation of the pressing member,
a partial outer circumferential portion being formed to cover a part of an outer circumferential surface of the shaft portion and including an outer circumferential surface slidable with respect to an inner circumferential surface of the cylinder portion and a proximal end surface having a helical shape and being slidable with respect to an inner end surface of the cylinder portion, and
the distal end portion, and
when the rotating operation of the shaft portion is caused by the operation lever, the pressing member moves along the direction of the axis of the cylinder portion while rotating around the axis by the proximal end surface of the partial outer circumferential portion sliding with respect to the inner end surface of the cylinder portion.

5. The endoscope according to claim 4, wherein the pressing member rotates substantially 90° around the axis of the cylinder portion while the valve mechanism is switched from the “open” state to the “closed” state.

6. The endoscope according to claim 3, wherein the distal end portion of the pressing member has an annular shape.

7. The endoscope according to claim 3, wherein the tube retaining portion includes a flat plate portion having a flat surface facing the distal end surface of the pressing member across the suction tube, and tube gripping portions formed on both sides of the flat plate portion.

8. The endoscope according to claim 7,
wherein at least a portion of the suction tube retained by the tube retaining portion extends inside the handle in parallel with the water tube,
the tube retaining portion includes a partition wall portion including a protruding plate extending in a direction perpendicular to the flat plate portion, and
the suction tube and the water tube are spaced apart from each other by the partition wall portion.
